(19) United States
(12) Patent Application Publication
(10) Pub. No.: US 2024/0218372 A1
(43) Pub. Date: Jul. 4, 2024

(54) COMPOSITION FOR TREATING DIABETIC RETINOPATHY CONTAINING scAAV2-shmTOR-SD (CRG-01)

(71) Applicant: CdmoGen Co., Ltd, Cheongju-si (KR)

(72) Inventors: KEERANG PARK, Cheongju-si (KR); STEVEN HYUN SEUNG LEE, Seoul (KR); JUN-SUB CHOI, Yongin-si (KR)

(73) Assignee: CdmoGen Co., Ltd, Cheongju-si (KR)

(21) Appl. No.: 18/536,423

(22) Filed: Dec. 12, 2023

Related U.S. Application Data
(60) Provisional application No. 63/432,061, filed on Dec. 13, 2022.

(51) Int. Cl. C12N 15/113; A61K 31/7105; A61P 27/02; C12N 15/86

(52) U.S. Cl. CPC C12N 15/1137; A61K 31/7105; A61P 27/02; C12N 15/86

(57) ABSTRACT

The present disclosure relates to a composition for treating diabetic retinopathy, containing scAAV2-shmTOR-SD (CRG-01), a therapeutic method of suppressing expression of mTOR proteins to treat diabetic retinopathy, and a method of using an RNAi method by means of short hairpin RNA (shRNA) as a method of suppressing expression and activity of mTOR proteins, and development of a composition for treating diabetic retinopathy by performing delivery to retina using AAV2 and being expressed in the form of mTOR shRNA as a method for expressing mTOR shRNA in cells of the retina.

### CROSS-REFERENCE TO RELATED APPLICATION

This application claims the benefit of U.S. Provisional Patent Application No. 63/432,061 filed on Dec. 13, 2022, in the U.S. Patent and Trademark Office, the entire disclosure of which is incorporated herein by reference for all purposes.

### SEQUENCE LISTING

This application contains a Sequence Listing submitted via Patent Center and hereby incorporated by reference in its entirety. The Sequence Listing is named SEQCRF_2280-488.txt, created on Dec. 11, 2023, and 12,288 bytes in size.

### BACKGROUND

### 1. Field of the Invention

The present disclosure relates to development of scAAV2-shmTOR-SD, a technology for inhibiting an mTOR protein using an AAV-delivered shRNA, and a gene therapeutic composition for treating diabetic retinopathy, which is a degenerative retinal disease.

### 2. Description of the Related Art

Diabetic retinopathy (DR) is a disease that occurs when capillaries in the retina are damaged due to high blood glucose, accompanied with a complication of diabetes that the retinal nerves degenerate with decreased vision. It accounts for a high proportion of blindness worldwide and occurs in approximately 90% of patients whose diabetes has progressed for 30 years or more, and its incidence reaches approximately 60-70% in the case that diabetes progresses around 15 years.

Diabetic retinopathy (DR), a leading cause of blindness, is becoming a major health concern worldwide, as diabetic retinopathy is expected to rise to a level of threatening eyesight in a quarter of the projected 592 million patients with diabetes mellitus (DM) by 2035. Diabetic retinopathy, which is a degenerative disease, begins with non-proliferative diabetic retinopathy (NPDR) at an early stage and then progresses to proliferative diabetic retinopathy (PDR), wherein the latter is characterized by neovascularization, a process induced by vascular endothelial growth factors (VEGFs). The co-occurrence of diabetic macular edema (DME) is a complex variable for the pathophysiology of diabetic retinopathy and treatment options thereof. It may occur during NPDR or PDR with thickened or swollen macula, which is the most common symptom of diabetic retinopathy.

Due to the most direct threat to vision of DR patients and a central role of VEGF, standard treatment for both PDR and DME is divided into laser photocoagulation and anti-VEGF therapies delivered via intravitreal injection. On the other hand, since a therapeutic agent for the protein-based intravitreal anti-VEGF therapies has a relatively short half-life, repeated administration is required to appropriately suppress VEGF in the long term. However, since these protein therapeutic agents require injections into the eyeball once every 1-2 months and cause serious side effects thereby, there is a great need to develop a fundamental therapeutic agent for diabetic retinopathy that has long-term therapeutic efficacy with a single injection.

### SUMMARY

### Problem to be Solved by the Invention

The present disclosure provides a pharmaceutical composition for preventing or treating diabetic retinopathy, including a recombinant vector including mTOR shRNA for mTOR inhibition.

### Means for Solving the Problem

An anti-VEGF-A therapy, which is a current therapy, may be burdensome for patients in terms of procedures and cost due to repeated intraocular injections and adversely affect patient compliance. It is also known that some of patients with diabetic retinopathy do not respond to these therapies. In order to achieve the above object, the present disclosure provides a therapeutic composition for treating diabetic retinopathy through inhibition of mTOR protein, which is associated with the onset and progression of the disease in patients with diabetic retinopathy, a composition of an AAV-based gene therapeutic agent showing a long-term effect through a single injection, and a therapeutic method.

### Effects of the Invention

The present disclosure relates to a composition for treating diabetic retinopathy including scAAV2-shmTOR-SD (CRG-01), which enables treatment of diabetic retinopathy by a single administration, thereby, compared to conventional therapeutic methods that require monthly intraocular injections, suppressing pain, eye damage, and infection in patients and reducing treatment cost.

### BRIEF DESCRIPTION OF THE DRAWINGS

FIG. 1 shows a structure of rAAV2-shmTOR-SD, an AAV2-based mTOR inhibitor for a gene therapy of diabetic retinopathy, and shmTOR shRNA sequences that may be loaded onto rAAV2-shmTOR-SD.

FIG. 2 shows graphs of mTOR expression inhibitory effects in accordance with a concentration of rAAV2-shmTOR-SD in HeLa cells.

FIG. 3 shows graphs of increased expression of mTOR and inhibited expression by rAAV2-shmTOR-SD in ARPE-19 cells cultured in a high-sugar growth medium.

FIG. 4 shows experimental designs to identify a therapeutic effect of rAAV2-shmTOR-SD used in the present disclosure on diabetic retinopathy and inhibition of mTOR expression in the retina of a mouse model of diabetic retinopathy by the administration of rAAV2-shmTOR-SD.

FIG. 5 shows images and analysis graphs of separated retinal blood vessels for observation of retinal vascular degeneration in a mouse model of diabetic retinopathy, showing the loss of pericytes and inhibited formation of acellular blood vessels in the retinal blood vessels by administration of rAAV2-shmTOR-SD compared to a control group.

FIG. 6 shows images and an analysis graph of observing the retinal vascular leakage using dextran-FITC in a mouse model of diabetic retinopathy, illustrating the inhibited vascular leakage by administration of a control virus vector and rAAV2-shmTOR-SD.

FIG. 7 shows images and an analysis graph of degenerative and protective effects of retinal tissues after staining the retinal tissue in a mouse model of diabetic retinopathy, wherein the retinal images of groups administered with a control virus vector and rAAV2-shmTOR-SD show that the thickness of the retinal tissue is thicker than that of the control group by administration of rAAV2-shmTOR-SD, suggesting a retinal neuroprotective effect.

FIG. 8 shows TUNEL staining images and an analysis graph for observation of retinal neuronal degeneration in a mouse model with diabetic retinopathy, wherein the retinal images of groups administered with a control virus vector and rAAV2-shmTOR-SD show that degeneration of retinal neurons is inhibited by administration of rAAV2-shmTOR-SD compared to the control group.

FIG. 9 shows images of NeuN staining for observation of retinal ganglion cells in a mouse model of diabetic retinopathy, images of GFAP staining for observation of glial cell activity, which is a marker of neuroinflammation in the retina, and images of detecting cell proliferation via GS staining, a Müller cell marker, analysis results of which are summarized in quantitative analysis graphs. Using retinal images of a normal control group (Normal), a control group with diabetic retinopathy (Sham (DR)), a rAAV2-shmTOR-SD test group, and a group administered with a rAAV2-shCon-SD test control group, an efficacy of inhibiting degeneration of retinal ganglion cells by administration of rAAV2-shmTOR-SD, compared to the control groups, was identified. In addition, an analysis result was obtained, suggesting that an increase in GFAP, which is characteristics of retinal neuroinflammation, was reduced by administration of rAAV2-shmTOR-SD.

FIG. 10 shows that expression of HIF-1α was suppressed by administration of rAAV2-shmTOR-SD compared to a control group, as a result of staining for HIF-1α in a mouse model of diabetic retinopathy and analyzing retinal images shown after administering several types of control groups and rAAV2-shmTOR-SD test groups.

### DETAILED DESCRIPTION

Therefore, the inventors have tried to develop a therapeutic agent capable of treating diabetic retinopathy via single administration and confirmed, using animal models, treatment of diabetic retinopathy by suppressing expression and activity for mTOR protein, a novel therapeutic target of diabetic retinopathy. As a result, the present disclosure was completed by finding that, when a gene therapeutic agent in which developed mTOR shRNA is packaged in an adeno-associated viral vector (AAV) is administered to model mice with diabetic retinopathy, macular edema symptoms, vascular leakage, and vascular degeneration, which are symptoms of diabetic retinopathy, were inhibited.

The present disclosure relates to a gene therapeutic agent for treating diabetic retinopathy, wherein AAV2-shmTOR-SD targeting mTOR proteins was developed to treat diabetic retinopathy, so as to treat diabetic retinopathy by suppressing expression of the mTOR protein.

It is known that expression of mammalian target of rapamycin (mTOR) increases in diabetic retinopathy induced by high blood sugar, and mTOR is known to be an important protein that is involved in various disease phenomena shown in the retina with diabetic retinopathy, such as neovascularization, retinal vascular degeneration, ischemic retinal nerve damage, free radical stress, inflammation, and immune response.

In the present disclosure, using shRNA designed to suppress expression of mTOR proteins, AAV2-shmTOR-SD, a novel therapeutic agent, was developed by mounting the same on a viral vector for continuous expression in the retina to derive a therapeutic effect, and the therapeutic effect was identified in the therapeutic efficacy test using cells and animals.

On the other hand, diabetic retinopathy is a very multifactorial disorder that involves various processes contributing to progression of diabetes-related hyperglycemia, oxidative stress, inflammation, and systemic effects on retinal blood vessels due to hypoxia. These factors are associated with factors that promote DME development, especially angiogenesis and Müller cell activity. Therefore, therapeutic strategies that focus on targets with broader effects such as mammalian target of rapamycin (mTOR) may be more effective than currently available treatment options to cope with DR and DME progression and various aspects of pathophysiology. mTOR, a serine/threonine kinase, which is involved in a number of cellular metabolic processes and signaling pathways, has been identified as a potential therapeutic target for a number of angiogenic retinal diseases, including PDR, while mTOR is involved in a variety of ocular dysfunctions.

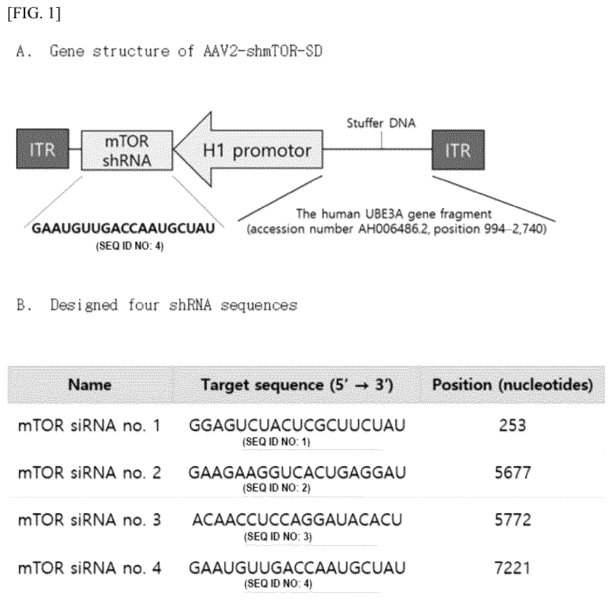

In addition, gene therapies that derive long-term effects without frequent intravitreal injections will overcome issues on the half-life and repeated injections, which are the main limitations of widely used anti-VEGF drugs, especially those that are suitable for ophthalmic diseases. Adeno-associated virus (AAV) vectors, the gene carrier vectors used in the development of this therapeutic agent, are non-pathogenic and highly safe, and excellent infectivity and transduction efficiency for a wide range of tissues and cells, including dividing and non-dividing cells, were demonstrated, proving the advantages in that, once infected, it remains episomally to expresses the introduced gene for several years, Luxturna, for example, a product developed and approved as an AAV-based gene therapeutic agent. Therefore, it is being used very appropriately in the development of therapeutic agents for diseases such as therapeutic agents for macular degeneration, which causes blindness among eye diseases, therapeutic agents for diabetic retinopathy, and therapeutic agents for retinitis pigmentosa, which causes blindness due to certain gene mutations. Based on the characteristics of these AAV vectors, the therapeutic vector of the present disclosure was designed, with the scAAV2 vector including a short hairpin RNA that inhibits mTOR expression, and for the completeness in the viral particle packaging, the scAAV2-shmTOR-SD or rAAV2-shmTOR-SD vector was constructed in addition to stuffer DNA sequences, as shown in FIG. 1A.

Then, the inventors identified mTOR expression and an ability of inhibiting increased mTOR expression by rAAV2-shmTOR-SD in an animal model of diabetic retinopathy. In the present disclosure, it was found that rAAV2-shmTOR-SD effectively reduces mTOR expression by delivery via intravitreal injection. In addition, in regard to the pathophysiological characteristics of diabetic retinopathy, the rAAV2-shmTOR-SD viral vector for treating diabetic retinopathy has shown the effect of inhibiting or protecting the pericellular cell loss, acellular capillary formation, vascular leakage, and retinal neuronal degeneration, while showing anti-apoptotic effects. These results demonstrated the potential as the gene therapy for retinal vascular disorders, especially diabetic retinopathy.

The present disclosure provides a pharmaceutical composition for preventing or treating diabetic retinopathy, including a recombinant vector including mTOR shRNA for mTOR inhibition.

Preferably, the mTOR shRNA may be, but is not limited to, any one of shRNAs represented by SEQ ID NO: 1 to SEQ ID NO: 4.

Preferably, the recombinant vector may be an adeno-associated virus (AAV) and, more preferably, rAAV2 or another serotype rAAV, but is not limited thereto.

Preferably, the pharmaceutical composition may be administered via intravitreal injection, but is not limited to, to suppress expression and activity of mTOR in the retina.

As used herein, the term “vector” refers to a DNA product including a DNA sequence that is operationally linked to a suitable regulatory sequence capable of expressing DNA within a suitable host. Vectors may be plasmids, phage particles, or simply potential genome inserts. Once transformed into a suitable host, the vector may replicate and function independently of the host genome, or, in some cases, integrate into the genome itself.

Since plasmids are currently the most commonly used form of vectors, the terms “plasmid” and “vector” are sometimes used interchangeably herein. For the purposes of the present disclosure, it is desirable to use plasmid vectors. A typical plasmid vector that may be used for this purpose has a structure that includes (a) a replication origin that allows replication to be carried out efficiently to include hundreds of plasmid vectors per host cell, (b) an antibiotic resistant gene that allows host cells transformed with plasmid vectors to be selected, and (c) a restriction enzyme cleavage site that allows foreign DNA fragments to be inserted. Even if an appropriate restriction enzyme cleavage site does not exist, vectors and foreign DNA may be easily ligated using synthetic oligonucleotide adaptors or linkers according to conventional methods. After ligation, the vector must be transformed into an appropriate host cell. Herein, the preferred host cell is a prokaryotic cell. Suitable prokaryotic host cells include E. coli DH5α, E. col JM101, E. coli K12, E. coli W3110, E. coli X1776, E. coli XL-1Blue (Stratagene), E. coli B, and E. coli B21. However, E. coli strains such as FMB101, NM522, NM538, and NM539, as well as other prokaryotic species and genera, may also be used. In addition to the aforementioned E. coli, strains in the genus Agrobacterium such as Agrobacterium A4, bacilli such as Bacillus subtilis, another Enterobacteriaceae such as Salmonella typhimurium or Serratia marcescens, and various strains in the genus Pseudomonas may be used as host cells.

In the present disclosure, the composition for treating diabetic retinopathy may be prepared in an injectable formulation, and in the case of the injection formulation, it may be characterized by further inclusion of a stabilizer selected from the group consisting of ethyl oleate, polyvinylpyrrolidone (PVP10), ganglioside sodium L-lactate, zinc chloride, and sucrose, and in a preferred aspect, HEPES buffer (20 mM, pH 7.4) with 10 mg/ml ethyl oleate, 1 mg/ml PVP10, 0.1 mg/ml GM1, 1 mg/ml sodium L-lactate, 0.1 mg/ml zinc chloride, and 10 mg/ml sucrose may be used.

The carriers used in the pharmaceutical composition of the present disclosure include pharmaceutically acceptable carriers, supplements, and vehicles, and are collectively referred to as “pharmaceutically acceptable carriers.” Pharmaceutically acceptable carriers that may be used in the pharmaceutical composition of the present disclosure include, but are not limited to, ion exchange, alumina, aluminum stearate, lecithin, serum proteins (e.g., human serum albumin), buffers (e.g., various phosphates, glycine, sorbic acid, potassium sorbate, partial glyceride mixtures of saturated vegetable fatty acids), water, salts, or electrolytes (e.g., protamine sulfate, disodium hydrogen phosphate, potassium monohydrogen phosphate, sodium chloride, and zinc salts), colloidal silica, magnesium trisilicate, polyvinyl pyrrolidone, cellulose-based substrates, polyethylene glycol, sodium carboxymethylcellulose, polyacrylates, waxes, polyethylene-polyoxypropylene-blocking polymers, polyethylene glycol, and woolen fabric.

Hereinafter, the present disclosure will be described in more detail through example embodiments. These example embodiments are merely for the purpose of describing the present disclosure in detail, and it will be apparent to those skilled in the art that the scope of the present disclosure is not limited by these example embodiments according to the gist of the present disclosure.

### <Example 1> Preparation of rAAV2-shmTOR-SD

As shown in FIG. 1, therapeutic rAAV2-shmTOR-SD generated rAAV2-shmTOR-SD and rAAV2-shCon-SD [19] from the respective precursor plasmids, pAAV-shmTOR-GFP and pAAV-shCon-GFP. Briefly, inserted were an H1 promoter that regulates the expression of mTOR-inhibitory shRNA (5′-GAAUGUUGACCAAUGCUAU-3′; SEQ ID NO: 4) or control shRNA (5′-AUUCUAUCACUAGCGUGAC-3′; SEQ ID NO: 5) and a stuffer DNA sequence designed from the human UBE3A gene. All viral vectors used in this study were purchased from CdmoGen Co., Ltd. (Cheongju, Korea).

### <Example 2> Identification of Suppression of mTOR Expression in HeLa Cells

An inhibitory ability for expression of mTOR for rAAV2-shmTOR-SD, a manufactured therapeutic agent, was identified.

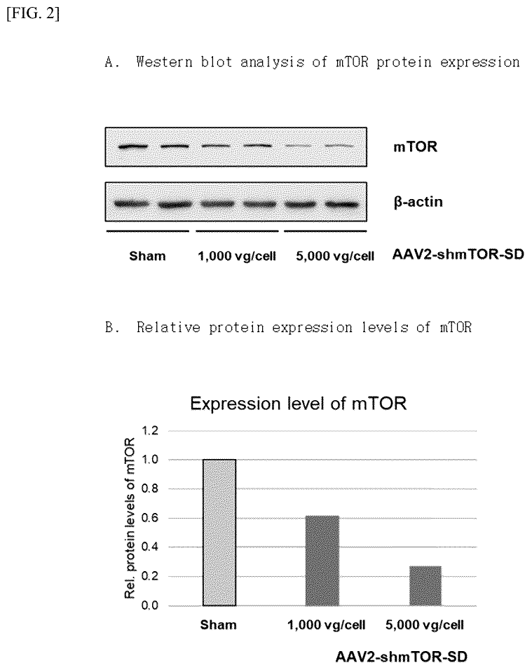

Cultured HeLa cells were infected with rAAV2-shmTOR-SD by concentration, mRNA and protein were extracted after 48 hours, and the expression of mTOR was detected. The results showed suppression of the mTOR expression according to the concentration of rAAV2-shmTOR-SD. For confirmation of expression of mTOR, mTOR mRNA expression was confirmed by the reverse transcription-PCR (RT-PCR) analysis and mTOR protein expression was confirmed by Western blot. As shown in FIG. 2, it was found that the expression of mTOR was suppressed by rAAV2-shmTOR-SD.

### <Example 3> mTOR Expression in Retinal Pigmented Epithelial Cell ARPE-19 Cultured in High Glucose Media

In diabetic retinopathy induced by persistent high blood glucose, all the cells constituting the retina are in an environment with high concentrations of glucose and affected by high concentrations of glucose. In the present disclosure, expression of mTOR in the retinal pigmented epithelial cells exposed to high glucose and down-regulation of mTOR expression by rAAV2-shmTOR-SD, a developed therapeutic agent, were detected.

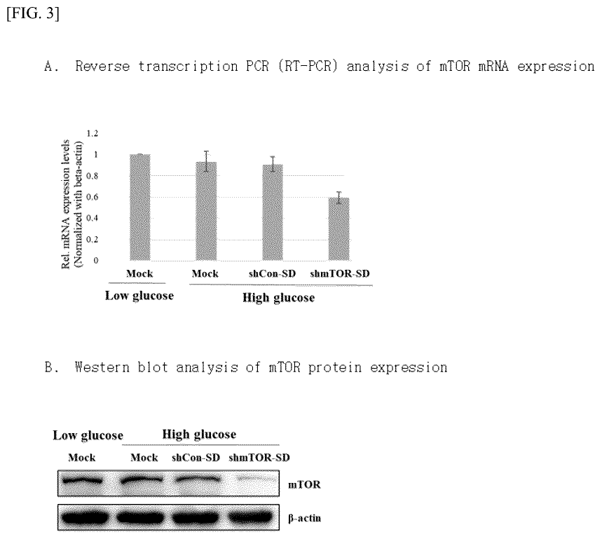

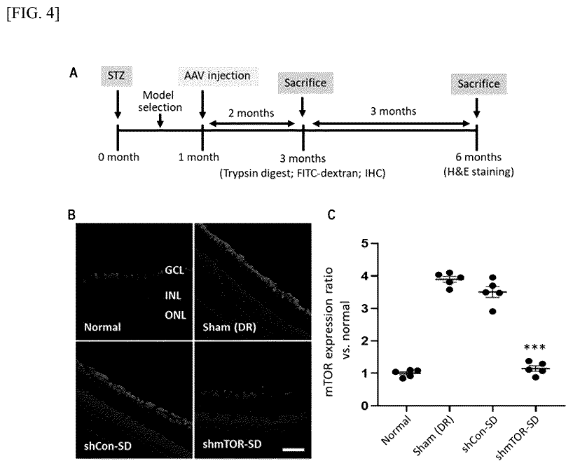

For retinal pigmented epithelial cells, ARPE-19 cells were used and cultured in a medium including high glucose (DMEM High glucose, DMEM high glucose, 11965092, Thermo Fisher Scientific), and the protein was extracted 48 hours after rAAV2-shmTOR-SD infection to detect expression of mTOR. In ARPE-19 cells, an increase in the high glucose-induced expression of mTOR was inhibited by rAAV2-shmTOR-SD. The results are shown in FIG. 3.

### <Example 4> Preparation of Mouse Animal Models of Diabetic Retinopathy

The mice used in the present disclosure were 7-week-old C57/BL6 (Orient Bio, Sungnam, Korea), and all animal care and experiments were conducted for ophthalmology and vision research in accordance with the Association for Research in Vision and Ophthalmology Resolution on the Use of Animals in Ophthalmic and Vision Research and supervised by the Animal Care and Use Committee of the University of Ulsan College of Medicine (Asan Medical Center, Seoul (Approval Number of Research plan: 2018-14-082, Approval Date: May 1, 2018)).

The experiment was carried out with 5 mice per group, under a controlled breeding environment at 25° C. and in a light and dark photoperiod of 12 hours during the test period, and the mice were allowed to freely intake water and food.

Hyperglycemic glycosuria was induced by single intraperitoneal injection of streptozotocin (STZ, 150 mg/kg, Sigma-Aldrich, St. Louis, MO), blood glucose was checked by Accu-Chek (Roche Diagnostics, Indianapolis, IN), and only mice with blood glucose levels greater than 300 mg/dL were selected to be used in the test.

### <Example 5> Intravitreal Injection of rAAV2-shmTOR-SD

One month after STZ induction, mice were anesthetized with a 4:1 mixture of Zoletil (zolazepam/tiletamine, 40 mg/kg, Virbac, Carros Cedex, France) and Rompun (xylazine, 5 mg/kg, Bayer Healthcare, Germany) by intraperitoneal injection, and Mydrin-P (0.5% tropicamide and 2.5% phenylephrine, Santen, Osaka, Japan) was applied to dilate the pupils for intraocular injection. Intravitreal injections of 1 μL (5.0×10⁷ v.g./μL/eye) of viral vector at a concentration of 5.0×10¹⁰ viral genomes (v.g.)/mL were performed into both eyes, and then the eyeballs were removed at 1, 2 or 5 months after injection.

The removed eyeballs were fixed in 4% paraformaldehyde, and then the fixed eyeballs were removed from the cornea and lens, embedded in Tissue-Tek (Miles Scientific, Napierville, IL) for cryosection in an eyecup form, and frozen at −80° C.

### <Example 6> Immunofluorescence Staining in Retinal Tissues

Ocular samples embedded in Tissue-Tek (Miles Scientific, Napierville, IL) were prepared as 5-10 μm thick cryotrans retinal sections, the sectioned sample was stained with anti-mTOR (AF15371; R&D Systems, Minneapolis, MN) to identify an in vivo efficacy of shRNA on mTOR inhibition, and retinal ganglion cells were immunostained using anti-NeuN (MAB377; Millipore, Burlington, MA). Anti-GFAP (12389; Cell Signaling Technology, Danvers, MA) or Anti-GS (MAB302; Millipore) was used for observation of retinal glial cell activity and cell proliferation via Müller cell markers. Retinal samples were reacted with the primary antibody at 4° C. for 12 hours, then washed in PBST three times for 10 minutes each, and reacted with the secondary antibody, Alexa Fluor 568 or 488 (Thermo Fisher Scientific, Waltham, MA), for 2 hours at room temperature. The cell nucleus was stained with DAPI (D9542, Sigma-Aldrich). Immunostained samples were observed using LSM 710 fluorescence confocal microscopy (Carl Zeiss Microscopy, Jena, Germany), and images were captured using the black edition of Zeiss Zen software (Carl Zeiss Microscopy) to be analyzed using ImageJ (National Institutes of Health, Bethesda, MD).

### <Example 7> Separation of Retinal Blood Vessels for Observation of Retinal Vascular Degeneration

In order to observe degeneration of retinal blood vessels in a mouse model of high blood glucose-induced diabetic retinopathy, the retinal blood vessels were separated, and changes in the retinal blood vessels and the degeneration of perivascular cells such as pericytes and endothelial cells and non-cellular blood vessels were observed. Separation of retinal blood vessels was performed via a trypsin digestion method (3% trypsin solution (15090046; Thermo Fisher Scientific) in 0.1 M Tris buffer (pH 7.8), 1 hour), and the separated vessels were stained with H&E to observe pericytes and acellular blood vessels.

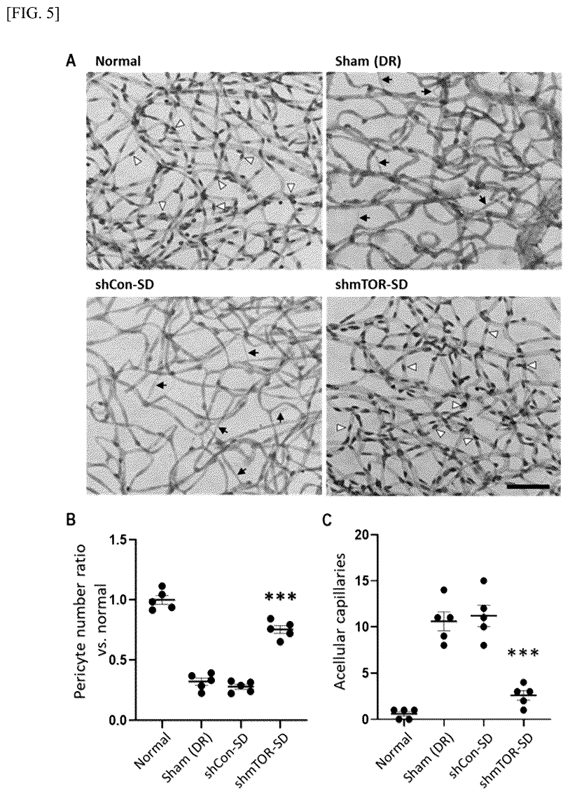

After intravitreal administration of rAAV2-shCon and rAAV-shmTOR-SD therapeutic vectors in the diabetic retinopathy model, as a result of observing blood vessels and perivascular cells, as shown in FIG. 5, vascular degeneration was inhibited by administration of rAAV-shmTOR-SD therapeutic vectors.

### <Example 8> Identification of Blood Vessel Leakage Inhibitory Effect in Diabetic Retinopathy Model Using Dextran-FITC

An animal model of diabetic retinopathy to observe the in vivo therapeutic effect of rAAV-shmTOR-SD prepared in Example 1 was produced as follows.

In mice (C57BL, 6-7 weeks of age), streptozotocin was prepared in 0.1 M citrate buffer at a concentration of 150 mg/kg to be injected once by intraperitoneal administration, 1 week after fasting for 8 hours and free feeding and drinking, blood glucose was measured to use as an animal model evaluated to have hyperglycemia (300 or higher by a blood glucose meter), rAAV-shmTOR-SD was administered into the both eyes at a dose of 5×10⁷ vg/μL/eye via intravitreal (IV) injection 4 weeks after streptozotocin injection, and the therapeutic efficacy on diabetic retinopathy was analyzed in disease animals induced with diabetes.

For analysis of the therapeutic effect, the retinal vascular leakage was analyzed by fluorescence fundus imaging, and for brief description, when it comes to the blood vessel leakage in the retina, the therapeutic effect in the control group and the group administered with therapeutic vector was comparatively analyzed by undergoing abdominal injection of dextran-FITC, removing the eyeball, separating the retina, spreading out on a glass slide, covering with cover glass, and observing with fluorescence microscopy.

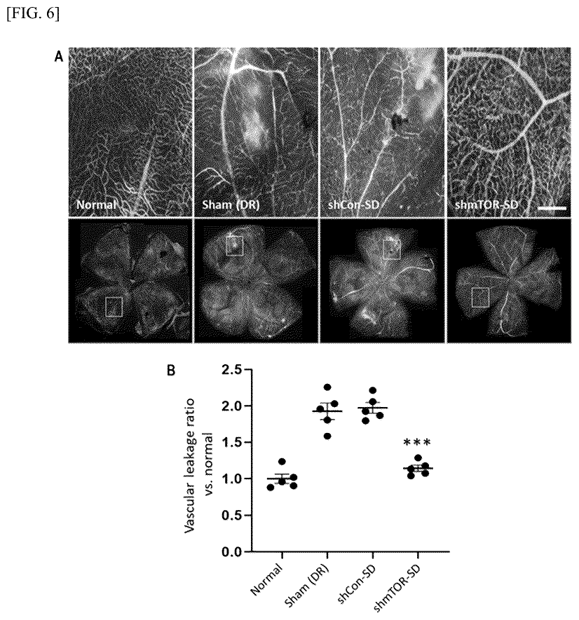

In the diabetic retinopathy model, as a result of detecting the vascular leakage by intravitreal administration of a control substance and rAAV-shmTOR-SD, as shown in FIG. 6, it was found that the vascular leakage was suppressed by administration of the rAAV-shmTOR-SD therapeutic vector.

### <Example 9> Retinal Tissue Staining for Observation of Retinal Degeneration

In order to observe degeneration of retinal blood vessels in a mouse model of high blood glucose-induced diabetic retinopathy, changes in retinal tissues were observed 6 months after disease induction. For staining of the retina, the eyeball was fixed, the lens and cornea were removed, a cryosection was prepared, and H&E staining was performed.

Stained retinal tissue slides were observed using a microscope, and changes in retinal tissues were analyzed by observing changes in retinal thickness. The retinal thickness was analyzed by measuring the change in the thickness of the inner nuclear layer and the inner plexiform layer.

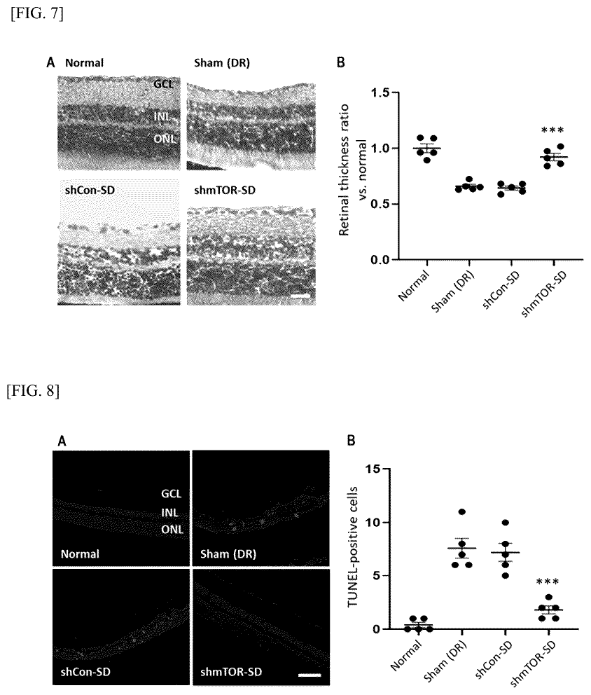

The results showed that degeneration of retinal nerve tissues was inhibited by administration of the rAAV-shmTOR-SD therapeutic vector, as shown in FIG. 7.

### <Example 10> Observation of Retinal Neuronal Degeneration in Model of High Blood Glucose-Induced Diabetic Retinopathy

For observation of degeneration of retinal neurons in the mouse model of high blood glucose-induced diabetic retinopathy, after injection of the therapeutic vector rAAV-shmTOR-SD which is a therapeutic vector, the eyeball was removed after a certain period of time, such as one month, and then frozen sections were prepared and observed. For observation of apoptosis, TUNEL staining was used to stain apoptotic cells.

The results showed that administration of rAAV-shmTOR-SD therapeutic vector had a therapeutic effect in potentially inhibiting retinal neuronal degeneration, as shown in FIG. 8.

### <Example 11> Observation of Preservation of Retinal Ganglion Cells, Glial Cell Activity, and HIF-1a in Mouse Model of High Blood Glucose-Induced Diabetic Retinopathy

In order to observe degeneration of the retina in a mouse model with of blood glucose-induced diabetic retinopathy, the survival of retinal ganglion cells and the activity of glial cells, a phenomenon of retinal neuroinflammation, were observed. For the detection of retinal ganglion cells via the method in Example 6, NeuN antibodies were used, and the observation of glial cells was performed using immunostaining with GFAP antibodies. Staining of NeuN and GFAP was performed using cryo-sectioned retinal tissues, and immunostaining was performed, followed by observation with LSM 720 confocal microscopy.

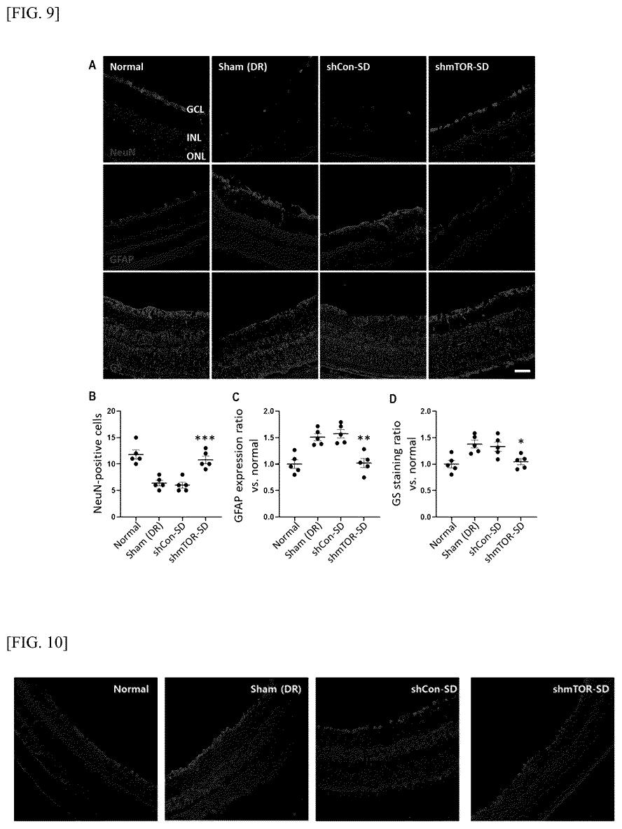

In the model of high blood glucose-induced diabetic retinopathy, as a result of observing the protective effect of retinal ganglion cells after intravitreal injection of 1 μL (5.0×10⁷ v.g./μL/eye) of rAAV-shmTOR-SD, as shown in FIG. 9, it was found that the retina of the Sham DR control group was reduced compared to that of normal control (Normal) in terms of NeuN staining, whereas preservation of NeuN-positive cells was observed by administration of rAAV-shmTOR-SD. In addition, in the staining of GFAP, which indicates inflammation of the retinal nerve, it was found that the activity of glial cells was suppressed by administration of rAAV-shmTOR-SD compared to the control group, indicating that the administration of rAAV-shmTOR-SD has the efficacy of suppressing inflammation in the retinal nerve.

HIF-1α is a well-known ischemia, oxidative stress marker in diabetic retinopathy and a protein known as a target for treating diabetic retinopathy. Using anti-HIF-1α as shown in FIG. 10 in the present disclosure, the expression of HIF-1α was confirmed in an animal model with streptozotocin-induced diabetic retinopathy (Sham (DR)), and it was found that expression of HIF-1α was suppressed by administration of rAAV2-shmTOR-SD.

As described above, while specific parts of the present disclosure are described in detail, for those skilled in the art, it is clear that the specific descriptions are merely preferred example embodiments and the scope of the present disclosure is not limited thereby. Accordingly, the substantial scope of the present disclosure will be defined by the appended claims and equivalents thereof.

What is claimed is:

1. A method of preventing or treating diabetic retinopathy, comprising:
administering a pharmaceutical composition comprising a recombinant vector comprising mTOR shRNA for mTOR inhibition to a subject.

2. The method of claim 1, wherein the mTOR shRNA is any one of shRNAs represented by SEQ ID NO: 1 to SEQ ID NO: 4.

3. The method of claim 1, wherein the recombinant vector is an adeno-associated virus (AAV).

4. The method of claim 1, wherein the recombinant vector is rAAV2 or another serotype rAAV.

5. The method of claim 1, wherein the pharmaceutical composition is administered via intravitreal injection to suppress expression and activity of mTOR in retina.


SEQUENCE LISTING (5 sequences)

SEQ ID NO: 1 - RNA, 19 nt, synthetic construct
  source 1..19: mol_type = other RNA
ggagtctactcgcttctat

SEQ ID NO: 2 - RNA, 19 nt, synthetic construct
  source 1..19: mol_type = other RNA
gaagaaggtcactgaggat

SEQ ID NO: 3 - RNA, 19 nt, synthetic construct
  source 1..19: mol_type = other RNA
acaacctccaggatacact

SEQ ID NO: 4 - RNA, 19 nt, synthetic construct
  source 1..19: mol_type = other RNA
gaatgttgaccaatgctat

SEQ ID NO: 5 - RNA, 19 nt, synthetic construct
  source 1..19: mol_type = other RNA
attctatcactagcgtgac